(19) United States
(12) Patent Application Publication
(10) Pub. No.: US 2025/0250537 A1
(43) Pub. Date: Aug. 7, 2025

(54) IN VITRO INSECT FAT CULTIVATION FOR CELLULAR AGRICULTURE APPLICATIONS

(71) Applicant: Trustees of Tufts College, Medford, MA (US)

(72) Inventors: Sophia Letcher, Medford, MA (US); Natalie Rubio, Medford, MA (US); David Kaplan, Medford, MA (US)

(21) Appl. No.: 18/875,537

(22) PCT Filed: Jun. 16, 2023
(86) PCT No.: PCT/US2023/068620

§ 371 Date: Dec. 16, 2024

Related U.S. Application Data
(60) Provisional application No. 63/366,532, filed on Jun. 16, 2022.

(51) Int. Cl. C12N 5/07; A23D 9/007; A23D 9/04

(52) U.S. Cl. CPC C12N 5/0601; A23D 9/007; A23D 9/04; C12N 2533/90

(57) ABSTRACT

Described herein are methods for making edible fat product using insect cells. The methods include isolating a population of insect cells, which may be precursor cells, seeding the population of insect cells onto a scaffold, and contacting the population of insect cells with a free fatty acid composition or a lipid composition. The cells seeded on the scaffold accumulate lipids, resulting in an edible fat tissue. Further disclosed are a composition of matter made from the method for making edible fat cells, a food product that includes the composition of matter, and an in vitro edible fat tissue comprising a population of insect precursor cells seeded on the scaffold.

### RELATED APPLICATIONS

This application claims the benefit of U.S. Provisional Patent Application Ser. No. 63/366,532, filed Jun. 16, 2022, which is fully incorporated by reference herein.

### FIELD OF THE INVENTION

This application relates to the production of edible cultured fat. Specifically, the production and/or accumulation of edible lipids within insect cells.

### BACKGROUND

Cultured meat technology has the potential to address a number of the public health, environmental degradation and animal welfare concerns associated with factory farmed meat. However, commercialization has been hindered by technical challenges related to cell sources, scale-up and the high costs of production. For example, cultured mammalian cells require growth at physiological temperatures, accumulate toxic by-products (e.g., lactate) at high rates, are generally sensitive to fluctuations in temperature and pH and require the supplementation of recombinant proteins and growth factors during serum-free culture. Besides muscle tissue production in cultured meat, fat is the other key tissue type essential to cultured meat production. In conventional (e.g., beef, pork, poultry) meat, adipose tissue is a key contributor to texture, flavor and nutrient profiles.

In the tissue engineering field, lipid accumulation (e.g., adipogenesis) in mammalian adipogenic precursor cells is generally achieved through medium supplementation of a hormonal cocktail containing insulin, dexamethasone and isobutylmethylxanthine. However, for cellular agriculture applications, less costly, food-grade medium supplements are preferable.

For cultured meat production, cells are often grown on biomaterial scaffolds to increase surface area for adhesion and to create structured and organized tissues. Useful scaffold systems need to be available in large quantities, edible and low-cost while supporting in vitro cultivation of the target cell types. Some plant-based biomaterials meet these criteria and have been proposed as potential scaffolds for cultured meat. For example, plants with high cellulose content were decellularized to remove plant genetic material, leaving behind naturally structured cellulose. Decellularized spinach, celery and grass supported myogenic cell adhesion and differentiation. Fat tissue does not require a strict architecture when compared to aligned muscle tissue, but it should benefit from a 3D environment for robust differentiation and to emulate the organoleptics of native fat. These efforts illustrate a need for cell cultured meat tissues, such as fat, which avoids the disadvantages currently associated with cultured mammalian cells and is organized in a structural manner such that the cultured fat tissue emulates the organoleptics of native meat fat.

### SUMMARY

The present disclosure provides for a method for producing an edible, cultured fat product that emulates the organoleptics of native meat fat. The cultured fat product may include cells that are able to produce and/or accumulate lipids, such as insect cells or insect precursor cells. The cultured fat product may be produced in vitro by seeding a population of insect cells or insect precursor cells onto a scaffold, wherein the scaffold is two- or three-dimensional, and contacting the population of insect cells or insect precursor cells seeded on the scaffold with a cell media containing a free fatty acid composition or a lipid composition, thereby accumulating lipids in the population of insect cells or insect precursor cells and producing therefrom an edible fat tissue. The cultured fat product may be produced in vitro by a method that includes (1) isolating a population of insect cells or insect precursor cells, (2) seeding the population of insect cells or insect precursor cells onto a three dimensional or two dimensional scaffold or in a two dimensional tissue culture plastic, for instance a well of a culture dish or container, (3) optionally cryopreserving and thawing the population of insect cells or insect precursor cells seeded on the scaffold, and (4) feeding the population of insect cells or insect precursor cells seeded on the scaffold with a free fatty acid composition or a lipid composition, thereby accumulating lipids in the population of insect cells or insect precursor cells and producing therefrom the edible fat tissue. A composition of matter made from the aforementioned method is further disclosed. A food product that includes the composition of matter made from the method is also disclosed. An in vitro edible fat tissue comprising a population of insect cells or insect precursor cells seeded on a scaffold is also disclosed. The population of insect cells or insect precursor cells may be enriched in lipid content via exposure to the free fatty acid composition or lipid composition.

The details of one or more embodiments are set forth in the accompanying drawings and the description below. Other features, objects, and advantages will be apparent from the description and drawings, and from the claims.

### DESCRIPTION OF THE FIGURES

FIG. 1 presents methods for the isolation of an embryonic cell line, scaffold fabrication, and in vitro lipid accumulation.

FIG. 2A-2C show culture and isolation of M. sexta embryonic precursor cells (Ms PCs). FIG. 2A: phase contrast image of Ms PC culture 14 days post-isolation. Some adherent and elongated cells are visible underneath the spherical cell aggregates. FIG. 2B: cell proliferation (P7) at increasing cell seeding densities over 5 days quantified by a fluorescence-based DNA quantification assay. Error bars are standard deviations, statistical significance is indicated by p-values (n=3, p-value<0.05). FIG. 2C: Phase contrast images of Ms PCs before and after cryopreservation (scale bar is 100 μm). Cells were passaged at day 5 post-cryopreservation.

FIG. 3 shows fluorescence microscopy images of striated muscle cells originating from M. sexta embryonic cell cultures from PO (top row) and P2 (bottom row). Cells were isolated, cultured for 2 months (PO) or 10 days (P2) and immunostained for myosin heavy chain (red) and counterstained for nuclei (blue). Scale bar is 100 μm.

FIGS. 4A-4D show fatty acid induced lipid accumulation in Ms PCs. FIG. A: fluorescence microscopy images of Ms PCs stained with BODIPY (green) and DAPI (blue) after 7 days in culture with growth media (control) or supplemented with Intralipid or oleic acid at 0.01, 0.1 or 1 mM (scale bar is 100 μm). FIG. 4B: fluorescence microscopy images of Ms PCs stained with BODIPY (green) and DAPI (blue) at day 1, 4, 7 and 14 after culture in growth media (control) or media supplemented with 0.1 mM Intralipid (scale bar is 100 μm). FIG. 4C: brightfield images of Ms PCs stained with Oil Red O (red) after 7 days in culture with growth media (control) or supplemented with Intralipid or oleic acid at 0.01, 0.1 or 1 mM (scale bar is 100 μm). FIG. 4D, lipid quantification assay from Oil Red O dye elution of day 7 Ms PC cultures. Error bars are standard deviations, statistical significance is indicated by p-values (n=3, p-value<0.05).

FIG. 5A-5B shows fatty acid induced lipid accumulation in Dm PCs. Panel 5A: fluorescence microscopy image of Dm PCs (green) stained with BODIPY (red) and DAPI (blue) after 7 days in culture with oleic acid (scale bar is 100 μm). Panel 5B: lipid quantification assay from Oil Red O dye elution of day 7 Dm PC cultures. Error bars are standard deviations, statistical significance is indicated by p-values (n=3, p-value<0.05).

FIG. 6A-6C shows M. sexta fat, in vivo and in vitro. Panel 6A: fluorescence microscopy images of cryosectioned in vivo fat body tissue from a 5th instar larva stained with BODIPY (green) or Phalloidin (pink) and DAPI (blue) (scale bar is 100 μm). Panel 6B: fluorescence microscopy images of cryosectioned in vivo fat body tissues from a 5th instar larva stained with Phalloidin (pink) and DAPI (blue) (scale bar=100 μm). Panel 6C: confocal microscopy image of an aggregate of Ms PCs after culture in 0.1 mM Intralipid for 14 days and stained for BODIPY (green) and DAPI (blue) (scale bar is 100 μm). Panel 6D: Quantification of neutral lipids in Ms PC samples cultured with or without 0.1 mM Intralipid for 7 days. Error bars are standard deviations, statistical significance is indicated by p-values (n=3, p-value<0.05).

FIG. 7 shows in vivo and in vitro lipidomics. Proportions of saturated (SFA), monounsaturated (MUFA) and polyunsaturated (PFA) fatty acids in Maduca sexta (M. sexta) at the wandering stage compared to Ms PCs untreated or treated with 0.1 mM Intralipid for 7 days and fat profiles reported for livestock animal tissue.

FIG. 8A-8G shows mycelium scaffolding. Panel 8A: sterilization screen of mycelium scaffolds by 70% ethanol, 10% bleach, autoclave or ethylene oxide gas. Scale bars are 1 mm. Panel 8B: scanning electron microscopy image of mycelia. Scale bar is 2 μm. Panel 8C: Dm PCs (green) growing in 2D on mycelium fibers dissociated with 4% bleach. Panel 8D: Dm PCs (white arrows) growing in 3D on live, mycelia fibers (red arrows). Panel 8E: untreated and decellularized mycelia cryosections (15 μm thick) stained with DAPI. Scale bars are 100 μm. Inset: photos of untreated and decellularized scaffolds. Panel 8F: cryosections of decellularized mycelia seeded with Ms PCs and cultured in control media or media supplemented with 0.1 mM Intralipid for 7 days before fixation and staining with BODIPY (green) and DAPI (blue). Scale bars are 100 μm. Panel 8G: comparison of elastic modulus of decellularized scaffolds without cells, with cells or with cells and treated with 0.1 mM Intralipid (all cultured for two weeks).

### DETAILED DESCRIPTION

It should be understood that this invention is not limited to the particular methodology, protocols, and reagents, etc., described herein and as such may vary as would be appreciated by a person of skill in the art. The terminology used herein is for the purpose of describing particular embodiments only and is not intended to limit the scope of the present disclosure. The inventions described herein are defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this disclosure pertains. Although any known methods, devices, and materials may be used in the practice or testing of the teachings herein, the methods, devices, and materials in this regard are described herein.

Cultured meat technology has the potential to address a number of the public health, environmental degradation and animal welfare concerns associated with factory farmed meat¹. However, commercialization has been hindered by technical challenges related to cell sources, scale-up and the high costs of production². Insect cell culture has been introduced as an advantageous platform for cultured meat production based on insect-specific bioprocess benefits that correlate to increased scalability and cost reductions³. Specifically, compared to most vertebrate cells, insect cells grow at near-ambient temperatures, accumulate toxic by-products (e.g., lactate) at lower rates, are generally more tolerant to fluctuations in temperature and pH and do not require the supplementation of recombinant proteins and growth factors during serum-free culture^{4,5}. Insect progenitor cells can be expanded in vitro and induced to differentiate into mature tissues for the generation of meat analogs⁶. Muscles have been cultured from Drosophila melanogaster (common fruit fly) and M. sexta (tobacco hornworm) embryonic cells by administering key hormones (e.g., juvenile hormone, ecdysone) in combination with scaffolds (e.g., silicone molds, chitosan films and sponges)⁷⁻⁹ to regulate proliferation and differentiation. Fat is the other key tissue type essential to cultured meat production¹⁰. In conventional (e.g., beef, pork, poultry) meat, adipose tissue is a key contributor to texture, flavor and nutrient profiles¹⁰. The analog of adipose tissue in insects is the fat body organ, which is an important site for lipid storage, metabolism, endocrinology and systemic immunity¹¹. The prominent cell type in fat body tissue is the trophocyte which stores lipids as triacylglycerols¹¹. Insect fat body cells have been cultured and characterized for decades but not with regard to food production applications^{6,12,13}.

Lipid accumulation (e.g., adipogenesis) in mammalian adipogenic precursor cells is generally achieved through medium supplementation of a hormonal cocktail containing insulin, dexamethasone and isobutylmethylxanthine¹⁰. For cellular agriculture applications, less costly, food-grade medium supplements are preferable. To that end, in vitro lipid accumulation in livestock cells has also been reported to be induced via free fatty acids (e.g., erucic acid, oleic), sourced from vegetable oils, at concentrations of 50-300 μM^{14,15}. Interestingly, free fatty acids (e.g., 400-1000 μM oleic acid) have also been reported to induce lipid accumulation in insect cell cultures (D. melanogaster embryonic cell lines) 16,17. For instance, treatment of S2 cells with 1 mM oleic acid over 24 hours resulted in a 5-fold increase in triacylglycerol content¹⁷.

For cultured meat production, cells are often grown on biomaterial scaffolds to increase surface area for adhesion and to create structured and organized tissues¹⁸. Useful scaffold systems need to be available in large quantities, edible and low-cost while supporting in vitro cultivation of the target cell types. Many plant-based biomaterials meet these criteria and have been proposed as potential scaffolds for cultured meat¹⁹. For example, plants with high cellulose content were decellularized to remove plant genetic material, leaving behind naturally structured cellulose¹⁸. Decellularized spinach, celery and grass supported myogenic cell adhesion and differentiation¹⁹⁻²¹. Furthermore, the native aligned structures of celery and grass induced myotube alignment^{19,21}. Fat tissue does not require a strict architecture when compared to aligned muscle tissue, but it should benefit from a 3D environment for robust differentiation and to emulate the organoleptics of native fat¹⁰. For this goal, mycelium (e.g., filamentous fungi) is a promising scaffold candidate. Mycelia are abundant, already consumed as a meat analogs and inexpensive to produce²². Mycelia are composed of interconnecting hyphal fibers that could encapsulate cells for three-dimensional culture²³. Decellularization may be helpful in removing fungal genetic material as to enhance the analysis of cell-scaffold constructs (e.g., imaging, DNA quantification)²⁰.

The present disclosure provides for an edible cell-cultured fat tissue (e.g., tissue made up of a collection of cells) and methods for making the fat tissue. The fat tissue is generated from lipid-producing and/or accumulating insect cells such as cultured precursor/embryonic insect cells. These cells may be induced to produce and/or store lipids. In some embodiments, the insect cells are seeded on a scaffold, providing structure to the fat product. The scaffold may be a natural product that includes mycelium. Together, the lipid-rich cells and the scaffold may produce a fat product that emulates animal fat in both taste and appearance.

In certain embodiments, the insect cell may include any cell or cell-type from the class Insecta. For example, the insect cell line may include any cell or cell-type from the Insecta orders, Lepidoptera, Hymenoptera, Coleoptera, Orthoptera, Odonata, Hemiptera, and Diptera. For instance, the insect cell or cell-type may include any species of insects from the order Lepidoptera (e.g., moths and butterflies) including but not limited to Maduca sexta (e.g., the tobacco hornworm).

In certain embodiments, the insect cell may of any cell type capable of accumulating lipids including but not limited to mycetocytes, oencytes, adipocytes, precursor cells, and cells of the fat body. Precursor cells are cells that are not entirely differentiated. Populations of insect precursor cells are readily found in eggs and other non-adult stages of insects.

In certain embodiments, the cells are grown in any cell media (e.g., growth media) capable of maintaining the growth or life of the cells. These cell media may include but are not limited to M3+BPYE growth media, Shields and Sang M3 media, Grace's insect media, Schneider's insect media, and SF9-based media. The cell media may include any supplementation including but not limited to serum (e.g., bovine, calf, fetal-bovine, goat), and antibiotics. For example, the cell media may include 10% fetal bovine serum.

In certain embodiments, the cells are contacted with a lipid composition. The lipid composition may include any lipid substance or lipid containing substance including but not limited to oleic acid, erucic acid, and Intralipid. Intralipid is a sterile, non-pyrogenic fat emulsion prepared for intravenous administration as a source of calories and essential fatty acids. In one form, Intralipid includes 20% Soybean Oil, 1.2% Egg Yolk Phospholipids, 2.25% Glycerin, and Water. The lipid may be sourced from a plant source including but not limited to soybean oil, corn oil, or other oils produced by vegetables, grains, seeds, or legumes. In some embodiments, the lipid composition is an oil emulsion, such as a seed oil emulsion. The lipid may also be sourced from an animal. In some embodiments, the lipid composition includes cell media (e.g., growth media).

The cell media may include or be supplemented with between 0.001 mM to 100 mM of a lipid or lipid composition, between 0.01 mM and 10 mM of a lipid or lipid composition, or between 0.1 and 2 mM of a lipid or lipid composition. For example, the cell media may include or be supplemented with approximately 0.1 mM or approximately 1 mM lipid or lipid composition. The media may include or be supplemented with between 0.000032% (V/V) to 3.2% (V/V) of a lipid or lipid composition, between 0.00032% (V/V) and 0.32% (V/V) of a lipid or lipid composition, or between 0.0032% (V/V) and 0.064% (V/V) of a lipid or lipid composition. For example, the cell media may include or be supplemented with approximately 0.0032% (V/V) or approximately 0.032% (V/V) lipid or lipid composition.

In certain embodiments, the lipid profile of the accumulated lipids is controlled by the exposure of lipids or lipid mixtures/compositions to the cells under different conditions, such as lipid concentrations, lipid type, time of exposure, cell media selection, culture temperature, or other cell growth and cell culture characteristics. For example, a specific lipid type may be added to the cells based on a resultant predicted lipid profile known to be palatable (e.g., emulating the organoleptics of native meat fat), healthy or having other desired characteristics. In another example, the time of exposure of the lipid composition to the cells may be limited, as prolonged exposure may produce nonpalatable lipids.

In some embodiments, the addition of lipids or lipid compositions to the cell media result in an increase in intracellular lipid content. The increase in intracellular lipid content may range from a 10% to 1000% increase, a 20% to 500% increase, a 30% to 250% increase, a 40% to 200% increase, or a 50% to 100% increase as compared to a no-lipid control (e.g., by mass, concentration, or v/v). For instance, the addition of lipids to the cell media may result in an approximately 50% increase or approximately 1.5-fold increase in intracellular lipid content as compared to control (e.g., by mass, concentration, or v/v). The addition of lipids or lipid concentrations to the cell media may also result in increases in extracted cellular lipids comparable to the ranges and approximations stated above.

As used herein, the term “enriched” is defined as an increase in the concentration or amount of material from a control or basal state. For example, the addition of lipids or lipid compositions to the insect cells may effectively increase, or enrich, the cells with lipids more than a no-lipid control. For instance, the addition of lipids or lipid compositions to the cells may enrich the cells with lipids with ranges and approximations as stated above.

In certain embodiments, the insect cells contacted by the lipid or lipid composition may become enriched with specific lipid species. These enriched lipid species may include but not be limited to polyunsaturated fatty acids (PUFAs), octadecenoic acid (18:1), eicosenoic acid (20:1), octadecadienoic acid (18:2), and alpha linoleic acid (18:3). The lipids accumulated by the cells may include any lipid listed in Table 2.

As used herein “fat tissue” is defined as the insect cell-containing structured product of this disclosure. The term “fat product” is defined as any product that includes, in some form, the fat tissue. The fat product may include either the fat tissue as the structured product, or the fat tissue as a disseminated or processed product. For example, the fat product may include fat tissue that has been processed in a blender.

In certain embodiments, the fat tissue may be included within a composition of matter including but not limited to a food product. The food product may include any edible product that typically includes lipids, particularly edible food products that traditionally have the look and/or taste of animal fat. For example, the fat tissue may be incorporated into a steak (e.g., a prepared bovine meat product). In another example, the fat tissue may be incorporated into a steak that is intended to look and/or taste like, but does not include, an animal product, such as a steak, where the muscle portion is made of plant or other material. The food product containing fat tissue may also emulate processed lipid-containing animal products such as lard, tallow, and butter. The food product may also be used in the cooking and processing of other foods. For example, the food product may include the fat tissue that has been used in the cooking/frying of vegetables.

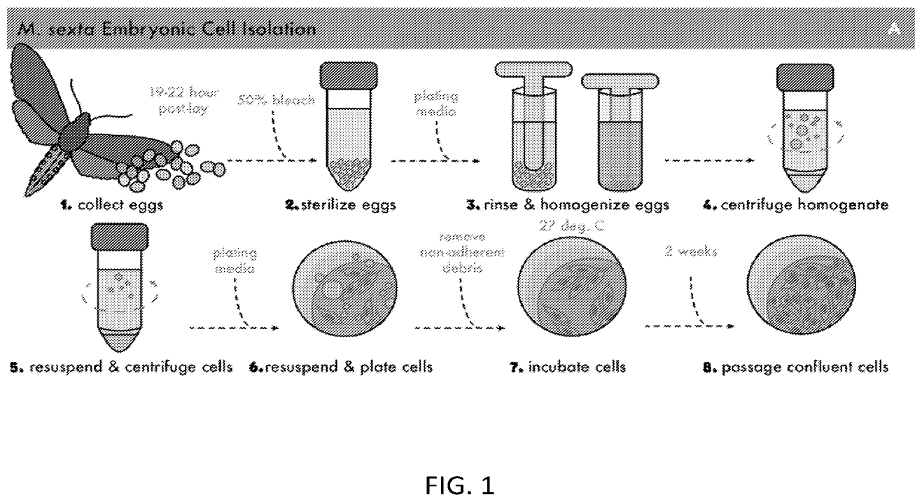

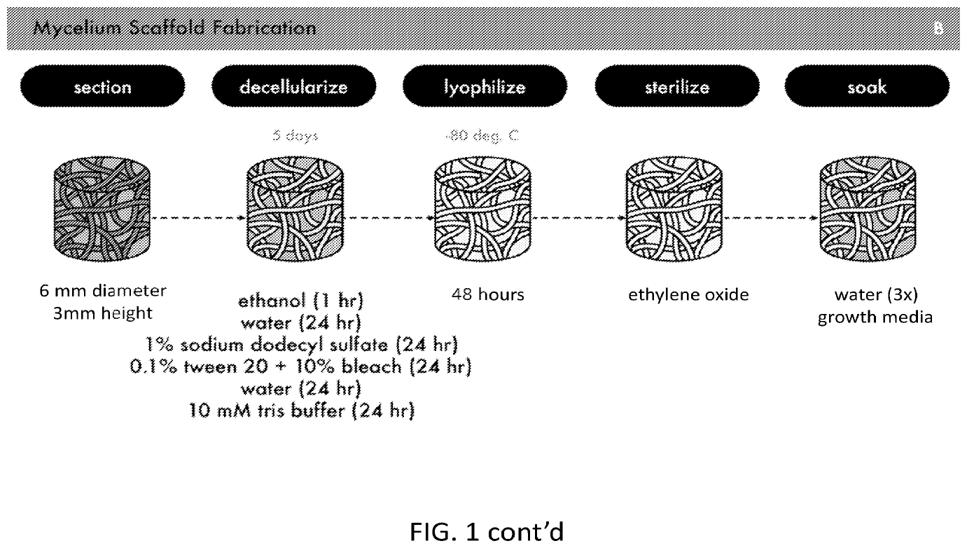

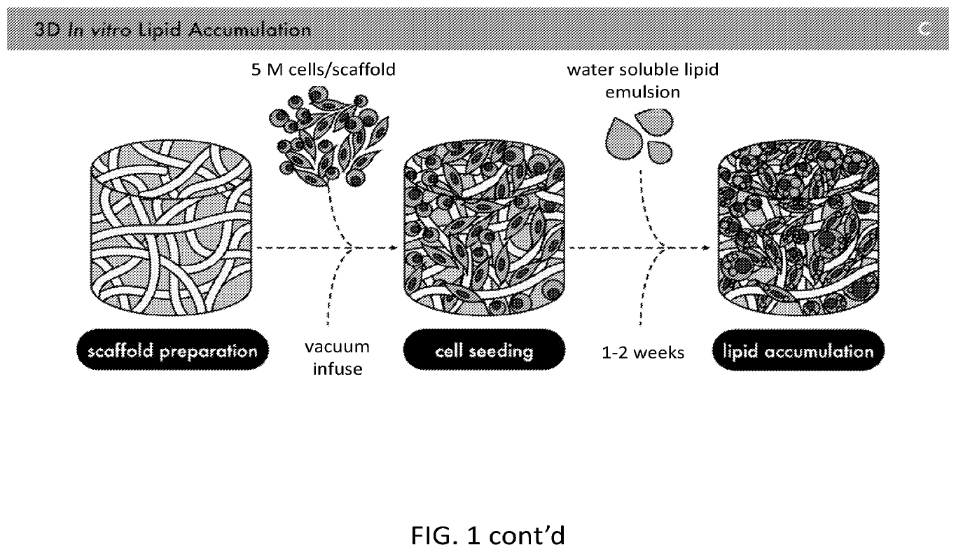

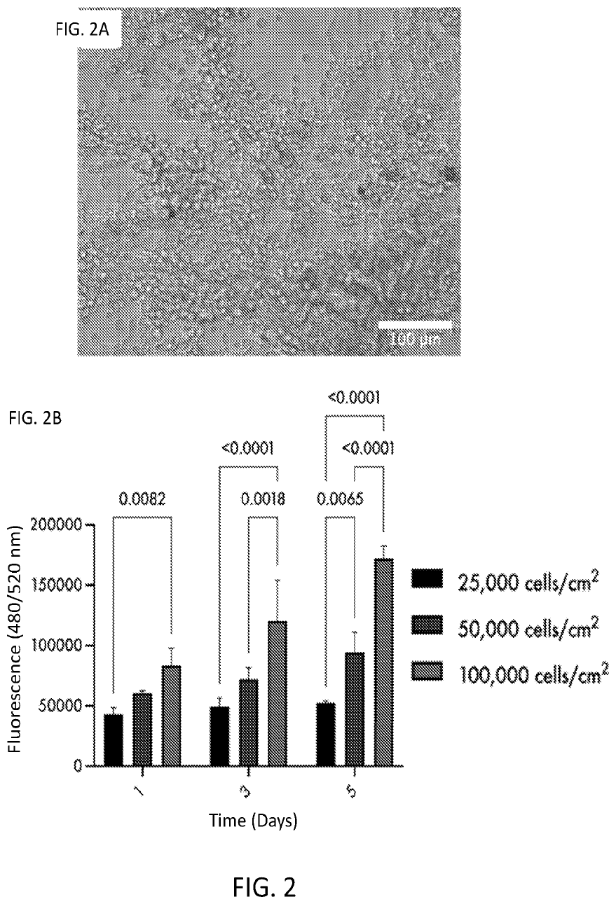

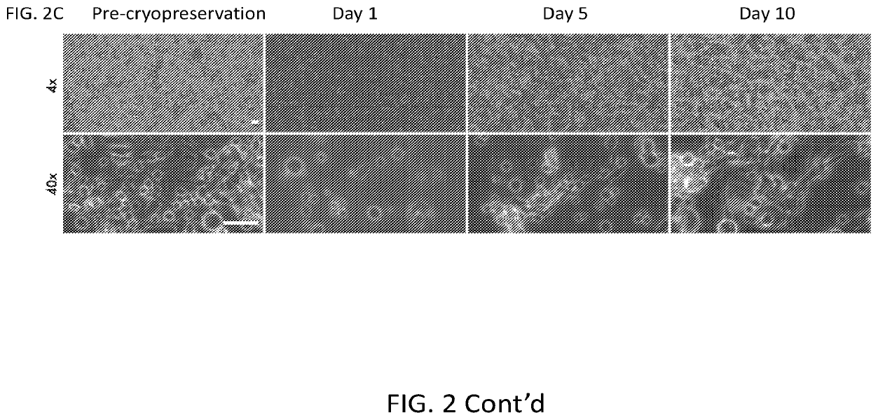

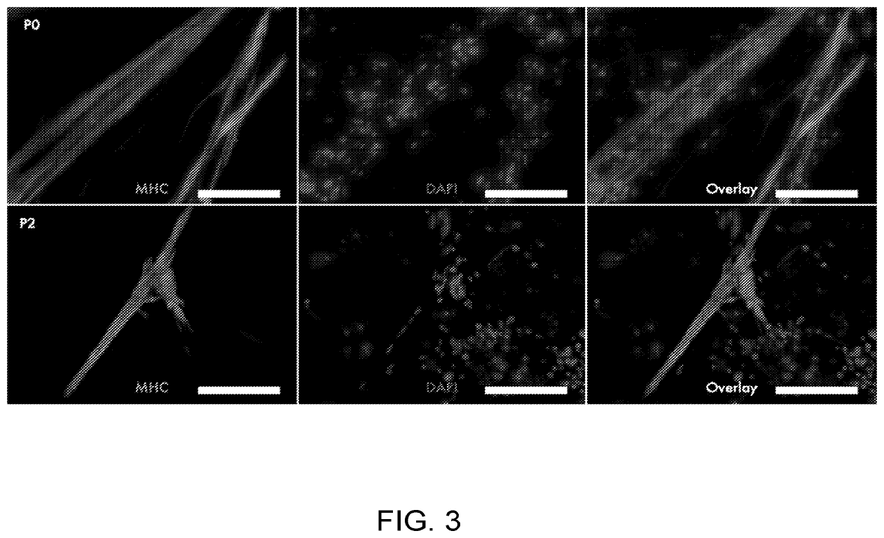

Methods for insect cell isolation and scaffold fabrication are described below and as illustrated herein, see, e.g., FIG. 1. In certain embodiments, embryonic precursor cells are isolated from M. sexta eggs using protocols adapted from previous work (Baryshyan, A. L. Et al., Isolation and Maintenance-Free Culture of Contractile Myotubes from Manduca sexta Embryos. PLOS ONE 7, e31598 (2012)), which is incorporated by reference in its entirety. A few days after the isolation, a subpopulation of the cells forms networks of contractile muscle. After two weeks, the cultures are characterized by the presence of three predominant populations: (1) proliferative, loosely adherent, aggregated or suspended spherical cells (FIG. 2, A), (2) adherent, elongated cells forming a monolayer on the surface of the culture vessel and (3) networks of contractile muscle cells that stained positive for myosin heavy chain (FIG. 3). The first population is passaged by sloughing and transferred into fresh culture vessels where the cells proliferate and are passaged approximately once a week. Upon passaging, subsets of cells adhere and elongate, while others remained aggregate and spherical. Stable growth rates are observed until approximately passage 15, when cells continue to appear healthy but grow notably slower. The adherent populations were passaged by trypsinization or cell scraping and were transferred into fresh culture vessels, where growth was observably slower relative to the first population. Robust and persistent (e.g., multiple weeks) cell network contractions were observed in the initial isolations, while contractile networks were less frequently observed in the subcultured populations. The method may include thawing cryopreserved insect precursor cells onto the scaffold and harvesting and/or cryopreserving cells from the scaffold.

As used herein, “contacting” is defined in a cellular setting as bringing two entities, such as a cell and a lipid composition, together so that the entities may interact (e.g., the cell may accumulate lipids from the lipid composition). Contacting in this setting may then mean “touching” or “nearly touching.” In some instances, contacting of a lipid composition to the cell may be also referred to as “feeding” (e.g., feeding a cell with the lipid composition).

The population of proliferative, spherical cells used for subsequent experiments are referred to as Ms PCs (M. sexta precursor cells). These cells were selected based on their growth rate, ease of passaging, homogenous morphology and loose adherence-indicating potential for future adaption to suspension culture⁷. In an example experiment, a growth curve for Ms PCs was performed to determine the optimal seeding density for propagation (FIG. 2, B). Adherent mammalian cells are generally seeded at 5,000 cells/cm² but insect cells are relatively smaller (e.g., Ms PCs are ˜5 times smaller in volume than C2C12s) and often seeded at a much higher density (e.g., 50,000 cells/cm²)^{7,24}. Over 5 days, the average specific growth rates for Ms PCs seeded at 25,000, 50,000 and 100,000 cells/cm² were 0.0017, 0.0037 and 0.006 hours-1, respectively. Based on this result, 100,000 cells/cm² were used as the seeding density for subsequent experiments. After the Ms PCs had been expanded to passage 4, they were cryopreserved at high density (i.e., 9-10E6 cells/mL) in growth media supplemented with fetal bovine serum (10-30%) and dimethyl sulfoxide (10%) (FIG. 2, C). Following thawing, cryopreserved cells were initially less viable (75% viability compared to 86% prior to cryopreservation), but rapidly recovered after 5 days (91%) (Table 1). After 10 days, the recovered cells looked morphologically similar to the initial cell populations and continued to grow at near-equivalent rates (reaching ˜90% confluency once a week). Ms PCs were also tested for mycoplasma contamination. In certain embodiments, the insect cells are seeded at a range of from at or about 20,000 cells/cm² to at about 250,000 or more cells/cm². The two-dimensional seeding density may be at least one cell/cm², at least 15,000 cells/cm², at least 20,000 cells/cm², or at least 25,000 cells/cm² and at most 1,000,000 cells/cm², at most 500,000 cells/cm², at most 250,000 cells/cm², or at most 200,000 cells/cm², or at a range at or between the aforementioned values. The insect cells may be seeded at or at ranges including at or about one cell/cm², at or about 20,000 cells/cm², at or about 25,000 cells/cm², at or about 30,000 cells/cm², at or about 35,000 cells/cm², at or about 40,000 cells/cm², at or about 45,000 cells/cm², at or about 50,000 cells/cm², at or about 55,000 cells/cm², at or about 60,000 cells/cm², at or about 65,000 cells/cm², at or about 70,000 cells/cm², at or about 75,000 cells/cm², at or about 80,000 cells/cm², at or about 85,000 cells/cm², at or about 90,000 cells/cm², at or about 95,000 cells/cm², at or about 100,000 cells/cm², at or about 125,000 cells/cm², at or about 150,000 cells/cm², at or about 175,000 cells/cm², at or about 200,000 cells/cm², or at or about 250,000 or more cells/cm².

In certain embodiments, where the scaffold is three-dimensional, the seeding may be performed at a three-dimensional density that is a three-dimensional equivalent to the two-dimensional equivalent, such as the plating densities per square centimeter as stated above. For example, the three-dimensional seeding density may be equivalent to approximately at least 15,000 cells/cm³, at least 20,000 cells/cm³, or at least 25,000 cells/cm³ and at most 1,000,000 cells/cm³, 500,000 cells/cm³, at most 250,000 cells/cm³, or at most 200,000 cells/cm³, or at a range at or between the aforementioned values. The seeding three-dimensional scaffold may also be seeded at a density of at least 100,000 cells/cm³, at least 500,000 cells/cm³, or at least two million cells/cm³ and at most le9 cells/cm², at most le8 cells/cm³, or at most le7 cells/cm³, or at a range at or between the aforementioned values. The seeding may be performed at a three-dimensional seeding density that is at least one cell/cm³, at least 15,000 cells/cm³, at least 20,000 cells/cm³, or at least 25,000 cells/cm³ and at most 1,000,000 cells/cm³, at most 500,000 cells/cm³, at most 250,000 cells/cm³, or at most 200,000 cells/cm³, or at a range at or between the aforementioned values. The three-dimensional seeding density may also be a three-dimensional equivalent to the two-dimensional seeding density of at least one cell/cm², at least 15,000 cells/cm², at least 20,000 cells/cm², or at least 25,000 cells/cm² and at most 1,000,000 cells/cm², at most 500,000 cells/cm², at most 250,000 cells/cm², or at most 200,000 cells/cm², or at a range at or between the aforementioned values.

TABLE 1

| | Pre- | 24 Hours Post- | 5 Days Post- | 10 Days Post- |
|---|---|---|---|---|
| | Cryopres- | Cryopres- | Cryopres- | Cryopres- |
| | ervation | ervation | ervation | ervation |
| Passage No. | 4 | 5 | 5 | 6 |
| Doubling | <69 | — | <97 | <79 |
| Time | | | | |
| (hours) | | | | |
| Cell | 85.7 | 75.4 | 90.7 | 83.7 |
| Viability | | | | |
| (%) | | | | |
| Cell | 6 | 2 | 7 | 10 |
| Aggregation | | | | |
| (%) | | | | |

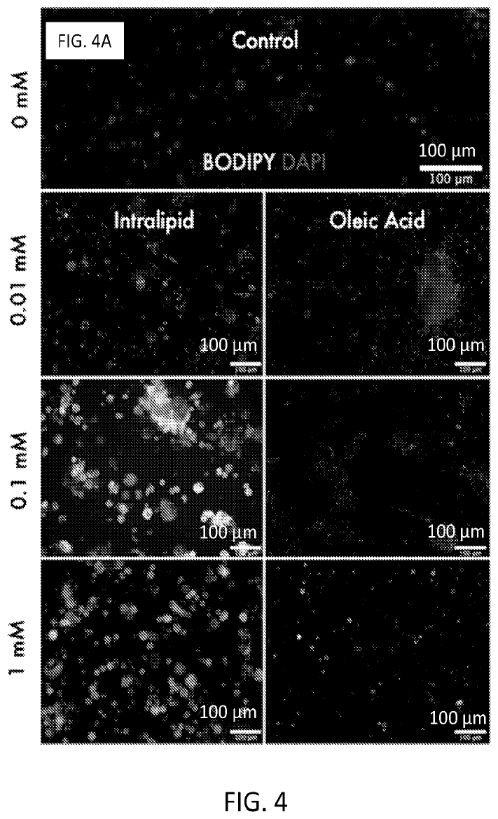

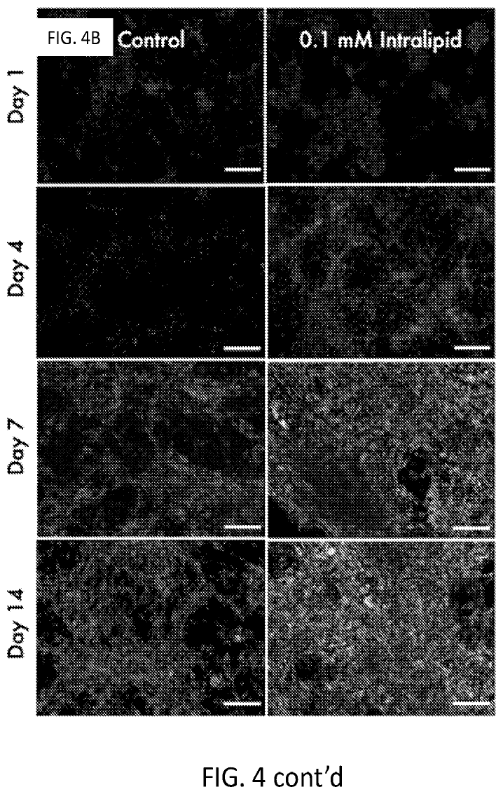

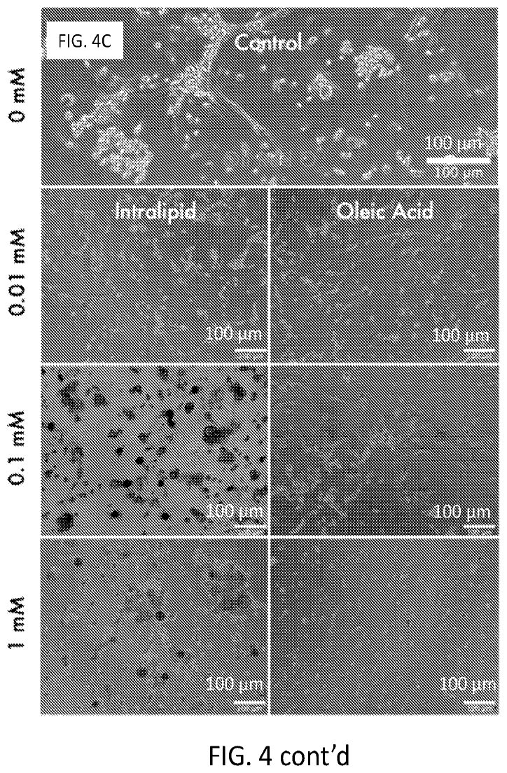

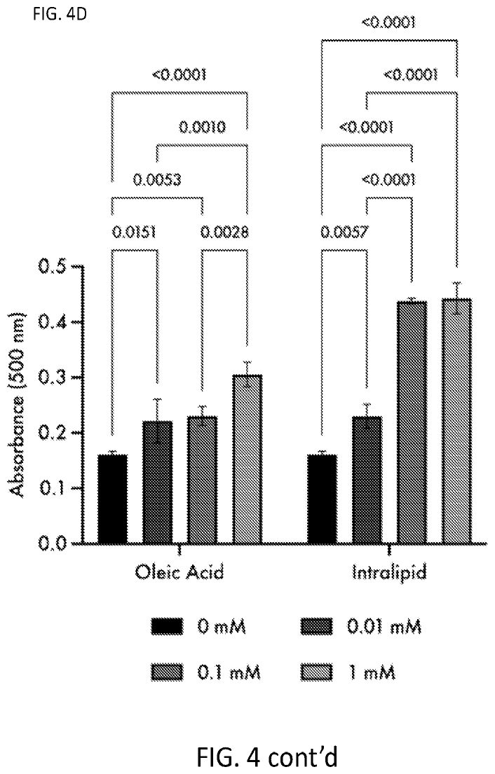

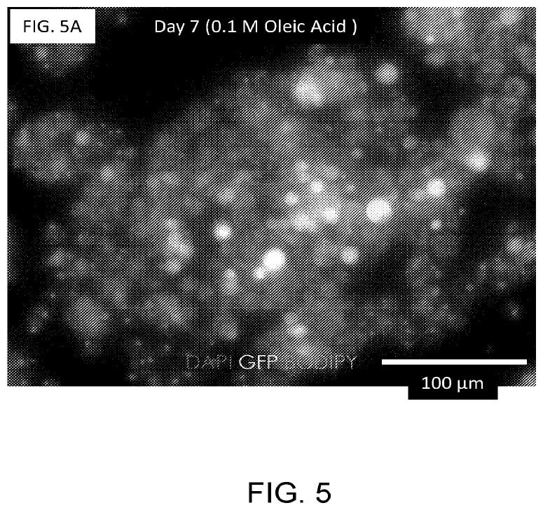

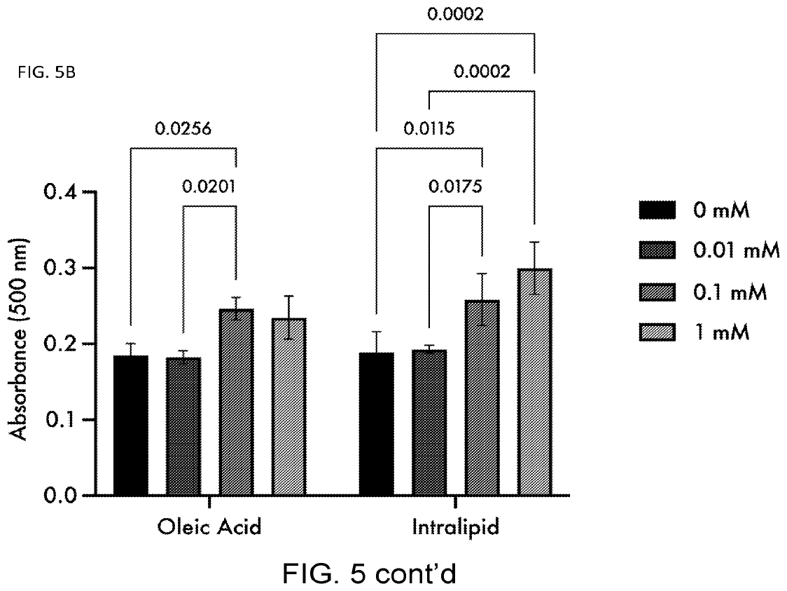

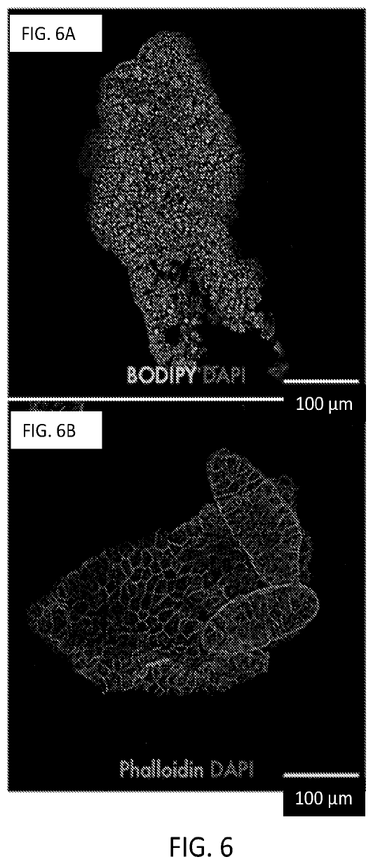

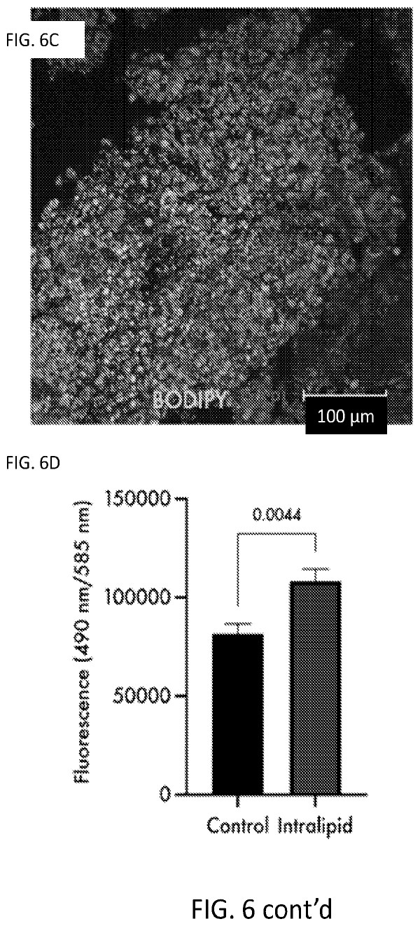

In certain embodiments, precursor cells are treated with a lipid composition to stimulate lipid production and/or storage. Vertebrate and invertebrate cells accumulate intracellular lipid droplets in response to fatty acid media supplementation^{14,17}. In an example experiment, Ms PCs were treated with different concentrations of fatty acids in the form of water-soluble oleic acid or Intralipid (e.g., soybean oil emulsion or other lipid composition). After 7 days in culture, lipid droplet formation was visible through BODIPY (FIG. 4, A) and Oil Red O (FIG. 4, C) staining in Intralipid conditions at 0.1 and 1 mM and oleic acid conditions at 1 mM, although the oleic acid treated cells exhibited unhealthy morphology, minimal growth, and were assumed to be nonviable. Lipid content was also quantified by eluting the Oil Red O dye and comparing absorbance levels (FIG. 4, D). The assay verified that Intralipid induced more lipid accumulation in the Ms PCs than oleic acid and that 0.1 and 1 mM were optimal concentrations of those tested. Similar trends were observed in D. melanogaster cells (FIG. 5, A, B). Lipid accumulation in Ms PCs via 0.1 mM Intralipid was observed over two weeks (FIG. 4, B), during which lipid accumulation steadily increased relative to the control cells. Neutral lipid content (e.g., triacylglycerols) was also quantified; treatment with 0.1 mM Intralipid for 14 days resulted in a significant increase (p=0.0044) in neutral lipid content in Ms PCs (FIG. 6, D). In certain embodiments, the lipid composition added to the insect cells and/or scaffold include any lipid containing substance including but not limited to a lipid emulsion, an oil emulsion, a seed oil emulsion, and fatty acid-containing solutions.

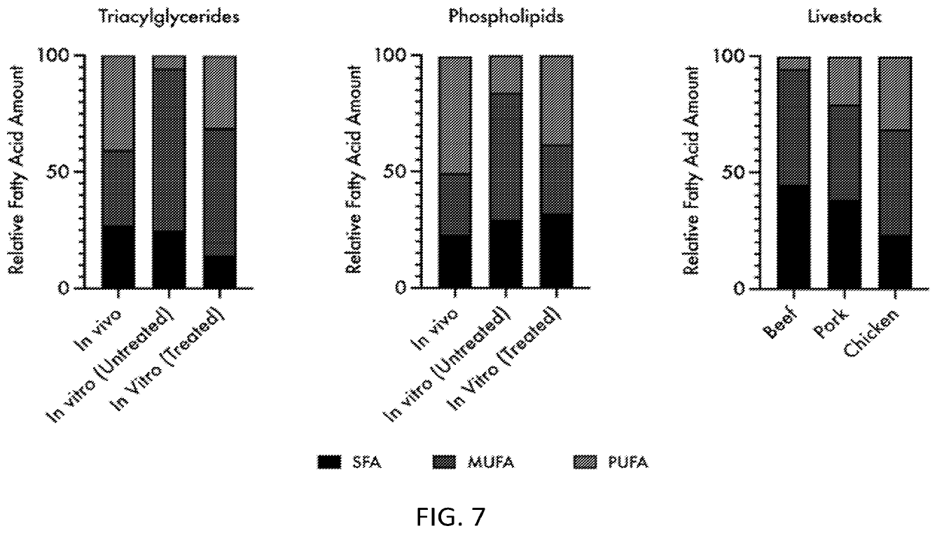

In certain embodiments, lipidomics is used to characterize fatty acids produced by the precursor cells. For example, lipidomics was used to characterize the fatty acid profiles in both untreated and treated Ms PCs (Table 2). Here, lipidomics data was split into proportions of saturated fatty acids (SFAs), monounsaturated fatty acids (MUFAs), and polyunsaturated fatty acids (PUFAs) for both phospholipids (important for cell membranes and taste) and triacylglycerols (the storage form of fat, making up most of lipids found in animals)²⁵⁻²⁷. In triacylglycerols, Intralipid treatment led to a shift from MUFA and SFAs to higher proportions of PUFA content (FIG. 7). It is important to note that the lipidomics data represents relative amounts of fatty acids rather than total or absolute amounts, and we estimated that Intralipid treated cells had approximately 30% greater triacylglycerol content than the untreated cells (FIG. 6, D). The decrease in SFA content was largely due to a decrease in palmitic acid (16:0), (the most common SFA in animals, plants and microorganisms) and a slight decrease in stearic acid (18:0) (Table 2)²⁸. Shifts in MUFAs was due to a large decrease in hexadecenoic acid (16:1), and slight increases in octadecenoic acid (18:1) and eicosenoic acid (20:1). PUFA changes were primarily due to an increase in octadecadienoic acid (18:2) (Table 2) 29. There was also a significant increase in alpha linoleic acid (18:3), an omega-3 fatty acid present in soybean oil (the primary component of Intralipid)³⁰. In the phospholipids, there was a relative decrease in MUFAs, increase in PUFA, and a slight increase in SFAs in the Intralipid treated conditions relative to controls. Again, octadecadienoic acid (18:2) accounted for a large amount of the increase in PUFAs (Table 2). When comparing the fatty acid content of Ms PC-generated fat and published data on traditional fat sources from livestock, in vitro Ms PC fat had a similar SFA content to chicken fat and lower SFA content compared with beef and pork fat³¹. Similarly, Ms PC-generated fat had a slightly higher PUFA content than chicken and substantially higher PUFA content than beef and pork. These fatty acid proportions were also similar to values previously published in whole M. sexta caterpillars³².

TABLE 2
Lipidomic Results for Ms PC Cells Untreated and
Treated with 0.1 mM Intralipid for 7 days Separated
by Phospholipids and Triacylglycerols.

| | | Untreated | | Treated | |
|---|---|---|---|---|---|
| | | Aver- | | Aver- | |
| Systematic | Omega | age | Standard | age | Standard |
| Name | Name | (%) | Deviation | (%) | Deviation |

Phospholipids
Saturated Fatty Acid

| | | | | | |
|---|---|---|---|---|---|
| dodecanoic acid | 12:0 | 0.01 | 0 | 0 | 0 |
| tetradecanoic acid | 14:0 | 0.26 | 0.06 | 0.09 | 0.03 |
| pentadecanoic | 15:0 | 0.19 | 0.01 | 0.37 | 0.01 |
| acid | | | | | |
| hexadecenoic acid | 16:0**** | 2.64 | 0.17 | 5.23 | 2.13 |
| heptadecanoic | 17:0 | 0.38 | 0.03 | 0.19 | 0.03 |
| acid | | | | | |
| octadecanoic acid | 18:0**** | 17.15 | 0.93 | 19.41 | 1.33 |
| heneicosanoic | 21:0 | 0.02 | 0 | 0.04 | 0.03 |
| acid | | | | | |
| docosanoic acid | 22:0 | 1.37 | 0.07 | 0.83 | 0.03 |

Monounsaturated Fatty Acid

| | | | | | |
|---|---|---|---|---|---|
| tetradecenoic acid | 14:1 | 0.93 | 0.13 | 0.09 | 0.04 |
| hexadecenoic acid | 16:1**** | 24.73 | 1.38 | 11.77 | 0.5 |
| heptadecenoic | 17:1 | 1.38 | 0.45 | 1.12 | 0.13 |
| acid | | | | | |
| octadecenoic acid | 18:1**** | 25.82 | 1.05 | 15.96 | 0.83 |
| eicosenoic acid | 20:1 | 0.28 | 0.02 | 0.11 | 0.01 |

Polyunsaturated Fatty Acid

| | | | | | |
|---|---|---|---|---|---|
| octadecadienoic | 18:2**** | 10.21 | 0.51 | 26.39 | 2.28 |
| acid | | | | | |
| octadecatrienoic | 18:3**** | 0.77 | 0.07 | 3.66 | 0.33 |
| acid | | | | | |
| eicosadienoic acid | 20:2* | 0.12 | 0.01 | 1.54 | 0.06 |
| eicosatrienoic | 20:3 | 0.6 | 0.03 | 1.33 | 0.17 |
| acid | | | | | |
| eicosatetraenoic | 20:4* | 0.71 | 0.1 | 2.12 | 1.98 |
| acid | | | | | |
| eicosapentaenoic | 20:5 | 0.17 | 0.08 | 0.2 | 0.03 |
| acid | | | | | |
| docosatetraenoic | 22:4 | 0.08 | 0.01 | 0.05 | 0.01 |
| acid | | | | | |
| docosapentaenoic | 22:5 | 0.25 | 0.08 | 0.31 | 0.06 |
| acid | | | | | |
| docosahexaenoic | 22:6 | 1.01 | 0.11 | 0.5 | 0.04 |
| acid | | | | | |

Triacylglycerides
Saturated Fatty Acid

| | | | | | |
|---|---|---|---|---|---|
| dodecanoic acid | 12:0 | 0.23 | 0.02 | 0.11 | 0.01 |
| tetradecanoic acid | 14:0 | 0.45 | 0.02 | 0.04 | 0.01 |
| pentadecanoic | 15:0 | 0.09 | 0.03 | 0.03 | 0 |
| acid | | | | | |
| hexadecenoic acid | 16:0**** | 12.25 | 0.45 | 3.09 | 0.21 |
| heptadecanoic | 17:0 | 0.41 | 0.17 | 0.63 | 0.07 |
| acid | | | | | |
| octadecanoic acid | 18:0**** | 7.22 | 0.48 | 4.63 | 0.63 |
| heneicosanoic | 21:0 | 0.26 | 0.02 | 0.54 | 0.3 |
| acid | | | | | |
| docosanoic acid | 22:0*** | 0.51 | 0.08 | 1.25 | 0.27 |

Monounsaturated Fatty Acid

| | | | | | |
|---|---|---|---|---|---|
| tetradecenoic acid | 14:1 | 0.12 | 0.01 | 0.05 | 0.01 |
| hexadecenoic acid | 16:1**** | 37.22 | 1 | 12.39 | 1.11 |
| heptadecenoic | 17:1 | 0.7 | 0.11 | 0.78 | 0.14 |
| acid | | | | | |
| octadecenoic acid | 18:1**** | 30.51 | 0.85 | 38.7 | 1.37 |
| eicosenoic acid | 20:1** | 0.39 | 0.11 | 1.79 | 0.35 |

Polyunsaturated Fatty Acid

| | | | | | |
|---|---|---|---|---|---|
| octadecadienoic | 18:2**** | 2.9 | 0.51 | 23.71 | 1.14 |
| acid | | | | | |
| eicosatrienoic | 20:3**** | 0.09 | 0.03 | 1.92 | 0.43 |
| acid | | | | | |
| eicosatetraenoic | 20:4 | 0.1 | 0.04 | 0.3 | 0.05 |
| acid | | | | | |
| eicosapentaenoic | 20:5 | 0.38 | 0.06 | 0.12 | 0.01 |
| acid | | | | | |
| docosatetraenoic | 22:4 | 0.04 | 0 | 0.05 | 0.04 |
| acid | | | | | |
| docosapentaenoic | 22:5 | 0.04 | 0.01 | 0.11 | 0.04 |
| acid | | | | | |
| docosahexaenoic | 22:6 | 0.5 | 0.04 | 0.51 | 0.08 |
| acid | | | | | |

p-values
*<0.0332;
**<0.0021;
***<0.0002;
****<0.0001

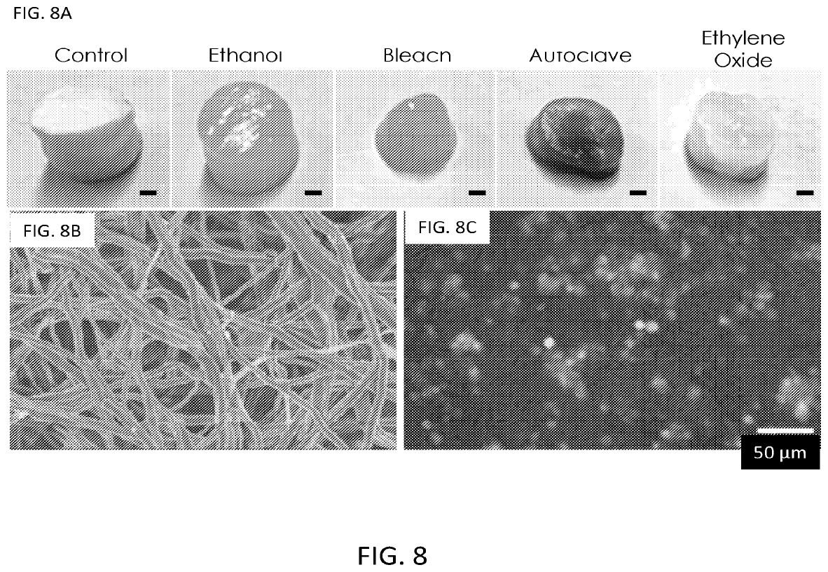

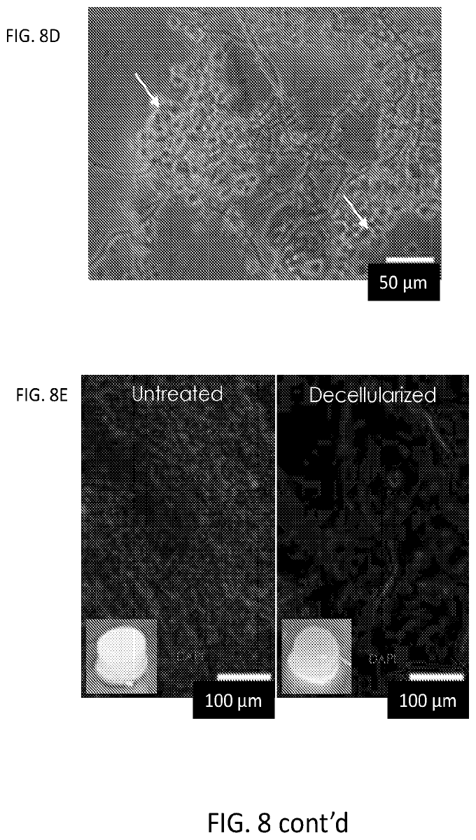

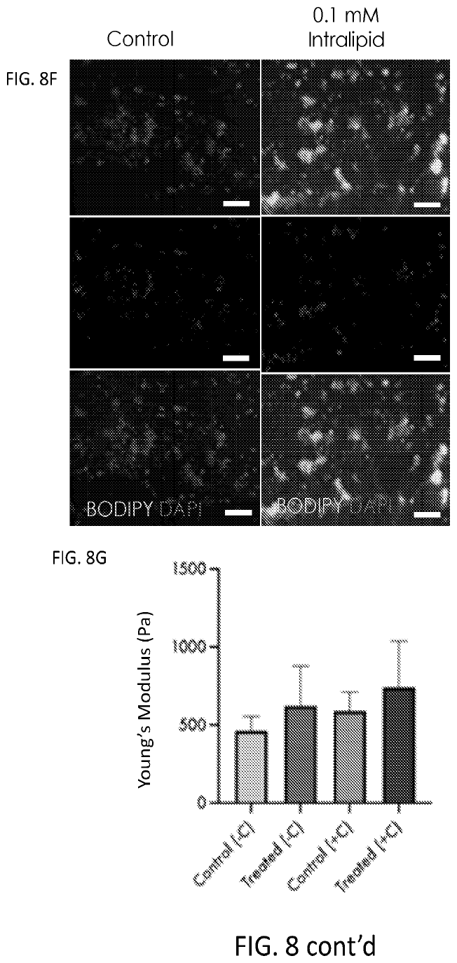

In certain embodiments, decellularized fungi, or fungi mycelium, is used as a scaffold (e.g., a decellularized mycelium scaffold) for seeded precursor cells. The decellularized mycelium was selected as a scaffold material based on the benefits of scalability, cost and established use in meat alternatives (e.g., Quorn)²². Decellularization may be performed by gentle rinsing in detergent solutions as indicated in FIG. 8 to eliminate fungal DNA (Table 3). Zeta potential, pore density, and pore size were also compared between untreated and decellularized scaffolds, showing that decellularization did not significantly impact scaffold surface charge or pore dimensions (Table 3). Mechanical properties were also investigated, and decellularized scaffolds had significantly lower elastic moduli compared with untreated scaffolds (Table 3). Prior to seeding cells into the scaffolds, an appropriate sterilization method was established by comparing four methods: treatment with 70% ethanol for 1 hour, treatment with 10% bleach for 1 hour, autoclave, or ethylene oxide treatment. Deformities of the scaffold occurred in the autoclave and bleach treatments (FIG. 8A). The autoclaved samples appeared burnt and shrunk in volume and the bleached samples were fragile and lost their original mass and shape. Ethylene oxide treatment was selected because scaffolds treated in this manner maintained structure, and this is also a sterilization method already established in the food industry³³. Before working with primary Ms PCs, preliminary experiments were performed with D. melanogaster precursor cells (Dm PCs) due to their rapid growth rate (e.g., 24-48 hour doubling time) and ease of imaging due to GFP-expression⁷. Dm PCs were initially cultured with dissociated mycelial fibers in 2D (FIG. 8, C), where the cells grew on top of and between the hyphae and remained viable based on GFP-expression. Dm PCs were also cultured on live (pre-sterilized) mycelium. The mycelium was viable in the insect cell culture media and began to grow, with the Dm PCs growing loosely associated with the hyphal fibers in 3D for a few days (FIG. 8, D). While initially an interesting result, the mycelium quickly outgrew the insect cells and overtook the culture. Once decellularization, sterilization and seeding methods had been optimized for mycelium scaffolds, Ms PCs were seeded at 5-6 million cells per scaffold. The Ms PCs infiltrated the scaffolds and dispersed throughout the constructs when seeded directly onto mycelium (i.e., passive seeding) (FIG. 8, F). After 14 days in lipid accumulation media (0.1 mM Intralipid as defined in 2D experiments), Ms PCs grown in 3D accumulated lipid droplets based on staining with BODIPY (FIG. 8, F). Mechanical properties of cell free, control (no Intralipid treatment) and 0.1 mM Intralipid treated 3D decellularized scaffold-cell systems were also investigated after 14 days through comparison of elastic moduli (FIG. 8, G). The addition of cells to scaffolds did not significantly impact the mechanical properties.

TABLE 3
Characterization of Untreated and
Decellularized Mycelium Scaffold

| | Untreated | Decellularized | Δ |
|---|---|---|---|
| Mass (mg/scaffold) | 5.2 ± 0.7^{a} | 3.4 ± 0.9^{a} | −35% |
| DNA (ng/mg) | 398 ± 49^{b} | 0 ± 0^{b} | −100% |
| Zeta Potential (mV) | −25.7 ± 1.1^{c} | −21.1 ± 2.2^{c} | +18% |
| Pore Density | 4292 ± 1440 | 3385 ± 758 | ns |
| (pores/mm²) | | | |
| Pore Size (μm²) | 89 ± 17 | 98 ± 25 | ns |
| Elastic modulus (Pa) | 1714 ± 693^{d} | 616 ± 246^{d} | −64% |

^{a}p-value 0.0005,
^{b}p-value 0.0050,
^{c}p-value 0.0317,
^{d}p-value 0.0103

In certain embodiments, the scaffold is sourced from fungal mycelium. The scaffold may include other fungi including but not limited to yeasts (i.e., Saccharomyces), and edible members of Chytridiomycota, Zygomycota, Ascomycota, Basidiomycota, and Glomeromycota. For example, the scaffold may be sourced from any fungi capable of producing mycelium.

In summary, M. sexta embryonic primary cells (Ms PCs) were isolated and a proliferative cell population was identified and expanded. Ms PCs were stimulated to produce lipids via culture with free fatty acids, and subsequent experiments showed that: (1) culture with 0.1 mM Intralipid (e.g., soybean oil emulsion) resulted in robust lipid accumulation over 7 days, (2) the cultured fat appearance and fatty acid composition mirrored in vivo M. sexta fat, (3) 0.1 mM Intralipid treatment resulted in a shift from relative SFA to PUFA content and an increase in linoleic acid, and (4) decellularized mycelial scaffolds supported lipid accumulation in 3D with mechanics suitable for fat-based foods.

Previously established primary cell cultures have been generated from M. sexta contractile muscle, but the isolated cells in the cultures did not proliferate over multiple passages and could not withstand cryopreservation⁸. The addition of primocin, a broad-spectrum antibiotic, during isolation and a focus on the proliferative, loosely adherent spherical cell population as opposed to the adherent and contractile muscle cell populations appear to overcome these limitations. Culture methods have also been developed for M. sexta midgut epithelial cells and cell lines from embryonic tissue have been previously characterized³⁴⁻³⁶. Previous work did not investigate M. sexta cells for food production or cultured meat. The present work shows an isolation method for generating a relatively homogenous cell population that consistently produces lipids upon stimulation with free fatty acids.

“Foodomics” describes a set of emerging analytical methods designed to assess the quality, nutritional value, and safety of foods by high-throughput methods such as metabolomics, proteomics, transcriptomics, and lipidomics³⁷. Lipidomics was used here to compare the fatty acid profile of Intralipid treated and untreated Ms PCs. Fatty acids are generally divided into four groups: saturated fatty acids (SFAs), monounsaturated fatty acids (MUFAs), polyunsaturated fatty acids (PUFAs), and trans fats³⁸. SFAs (e.g., butter, coconut oil) are comprised of carbon atoms that are all bonded to two hydrogen atoms. This composition makes SFAs very stable, but high levels of consumption lead to an increased risk of coronary heart disease and other negative health implications, resulting in recommendations to limit SFA intake³⁹⁻⁴¹. MUFAs are fatty acids with one pair of carbon atoms that have a double bond replacing the hydrogen-carbon bond. PUFAs have two or more carbon-carbon pairs and are generally considered healthier than SFAs. Increased intake of MUFAs and PUFAs is thought to lead to a decreased risk of coronary heart disease and may be important for the prevention and management of a variety of diseases^{42,43} Trans fats are manufactured through addition of hydrogen atoms to unsaturated oils and would not be present in Ms PC lipid data. The observed decrease in SFA and increase in PUFA content in Intralipid-treated triacylglycerols represents a shift to potentially “healthier” fat content. Proportions of SFA, MUFA, and PUFA in Ms PC fat were found to be similar to previously published reports of chicken fat³¹. Poultry is often hailed for being nutritionally favorable compared with red meat, a distinction primarily drawn from the differences in SFA and PUFA content⁴⁴. Another notable finding was the increase in linoleic acid (a PUFA) in triacylglycerols with 14-day Intralipid treatment. Preliminary studies show a range of benefits of replacing SFAs with linoleic acid, from reduced microglial inflammation in the brain to a preventative effect for coronary heart disease; however, the literature on linoleic acid benefits is controversial⁴⁵⁻⁴⁸.

Although Intralipid was used for lipidomics because lipid accumulation was observed to be more efficient compared with oleic acid, we expect that exploring other fatty acid treatments or concentrations may result in altered proportions of SFA, PUFA, and MUFAs. Thus, multiple products could be created from the same cell type using different fatty acid treatments to induce desired nutritional and flavor profiles.

In certain embodiments, the scaffold may be configured as a two-dimensional (2D) or near-2D structure. 2D scaffolds are relatively flat and are useful for generating thin-layered tissue constructs. 2D scaffolds may include living tissue or formerly living tissue, such as mycelium fibers or other fibers as described herein. The cells may also be plated or cultured as a 2D culture with or without a scaffold. For example, the cells may be cultured in a 2D tissue culture dish (e.g., a plastic-based tissue culture dish). Mycelium-based scaffolds may be decellularized and/or otherwise processed. For example, the mycelium-based scaffold may be processed so that DNA is removed. In some embodiments, the scaffold is edible. For example, the scaffold may include mycelium that can be safely ingested by a subject.

In certain embodiments, the scaffold may be configured as a three-dimensional (3D) structure. Although 2D cell culture is useful for pilot studies, generating large tissue constructs for food consumption will benefit from scaled-up production in three dimensions. Scaffolding materials for 3D applications may include materials such as mushroom chitosan, alginate, or cellulose³. In the present disclosure, Ms PCs were shown to be successfully cultured in a decellularized mycelial scaffold as indicated by presence of nuclei throughout the bulk of the construct. Notably, decellularized scaffolds had a lower elastic modulus when compared with untreated scaffolds. While decellularized scaffolds are still within the range of moduli for in vivo fat products⁴⁹, different strains of mycelia, fermentation methods or post-process treatments to stiffen the scaffolds could be explored if “tougher” products are desired. A major bottleneck in creating larger constructs of cultured meat is the difficulty of engineering in functional vascular systems to transport oxygen and nutrients throughout larger constructs. The natural mycelium matrix enables encapsulation of cells in 3D without the need for tissue engineered vasculature or microfabricated scaffolding, a major benefit towards scale-up in the future. Insect cells are also likely more adaptable to large-scale 3D culture because in vivo insects grow in the absence of a closed circulatory system.

While the current disclosure shows proof-of-concept results that Ms PCs can be used as a cultured fat source, future considerations include adaptation of Ms PCs to serum-free media and optimization of scaled-up growth of Ms PCs in suspension culture. D. melanogaster primary cells (Dm PCs) may be adapted to commercial serum-free medium, and a similar formulation would likely support Ms PC growth^{5,7}. Suspension culture is a useful method for scaled-up production of cultured meat because growth is not constrained by surface area. Ms PCs survived and proliferated in single cell suspension (data not shown) and cells grown in suspension were used for seeding the 3D scaffolds; however, suspension culture must be optimized to achieve growth rates that support large-scale production. While the research purpose here was to generate cultured fat, manipulation via other naturally occurring insect hormonal treatments (e.g., 20-hydroxyecdysone or juvenile hormone) may direct these cells towards alternate fates. It is envisioned that Ms PCs may be maintained as a “starter cell” population that can be used to produce multiple meat-relevant cell types beyond lipid production. Ms PC-produced fat could be used in various capacities: (1) combined with Ms PC muscle constructs to create combined muscle and fat products, (2) incorporated as a fat source in other cultured meat products (e.g., bovine, avian), or (3) used to provide animal-derived nutrition and fat taste in combined plant-based and cultured meat products.

Although traditional livestock species have also been shown to accumulate lipids upon treatment with fatty acids, Ms PCs are favorable for this approach due to their ease of growth, broader tolerance to environmental perturbations, and scale-up potential. This is demonstrated by their ability to grow at room temperature without carbon dioxide, antibiotic-free culture, adaptability to suspension and adherent cultures, and minimal medium changes.

In certain embodiments the disclosure relates to any of the following numbered paragraphs:
1. An in vitro method of making edible fat tissue, the method comprising:
   seeding a population of insect cells onto a scaffold, wherein the scaffold is two- or three-dimensional; and
   contacting the population of insect cells with a cell media containing a lipid composition, thereby accumulating lipids in the population of insect cells and producing therefrom an edible fat tissue.
2. The method of paragraph 1, wherein the population of insect cells is a population of precursor cells.
3. The method of paragraph 1 or paragraph 2, wherein the population of insect precursor cells is a population of Lepidoptera cells or Manduca sexta precursor cells.
4. The method of any one of the preceding paragraphs, wherein the scaffold is a fungal scaffold.
5. The method of paragraph 4, wherein the fungal scaffold is a decellularized Mycelium scaffold.
6. The method of any of paragraphs 1-5, wherein the scaffold is two-dimensional, and the seeding is performed at a two-dimensional seeding density of at least one cell/cm², at least 15,000 cells/cm², at least 20,000 cells/cm², or at least 25,000 cells/cm² and at most 1,000,000 cells/cm², at most 500,000 cells/cm², at most 250,000 cells/cm², or at most 200,000 cells/cm², or at a range at or between the aforementioned values.
7. The method of any of paragraphs 1-5, wherein the scaffold is three-dimensional and the seeding is performed at a three-dimensional seeding density that is a three-dimensional equivalent to the two-dimensional seeding density of at least one cell/cm², at least 15,000 cells/cm², at least 20,000 cells/cm², or at least 25,000 cells/cm² and at most 1,000,000 cells/cm², at most 500,000 cells/cm², at most 250,000 cells/cm², or at most 200,000 cells/cm², or at a range at or between the aforementioned values, or is at a three-dimensional seeding density of at least one cell/cm³, at least 15,000 cells/cm³, at least 20,000 cells/cm³, or at least 25,000 cells/cm³ and at most 1,000,000 cells/cm³, at most 500,000 cells/cm³, at most 250,000 cells/cm³, or at most 200,000 cells/cm³, or at a range at or between the aforementioned values.
8. The method of any one of the preceding paragraphs, further comprising the step of: cryopreserving and thawing the population of insect precursor cells seeded on the scaffold.
9. The method of any one of the preceding paragraphs, wherein a concentration of lipids in the cell media from the lipid composition ranges from 0.1 mM to 10 mM, wherein the lipid composition is an emulsion.
10. The method of paragraph 9, wherein the lipid composition is a seed oil emulsion, preferably a soybean oil emulsion.
11. The method any one of the preceding paragraphs, wherein the lipid composition and an exposure time are selected to produce a desired lipid profile in the edible fat tissue.
12. The method any one of the preceding paragraphs, further comprising the step of isolating a population of insect precursor cells.
13. A composition of matter made from the method of any one of the preceding paragraphs.
14. A food product containing the composition of matter of paragraph 13.
15. An in vitro edible fat tissue comprising a population of insect precursor cells seeded on a scaffold, wherein the population of insect precursor cells have been enriched in lipid content by exposure to a lipid composition.
16. The method of paragraph 15, wherein the population of insect precursor cells is a population of Lepidoptera cells.
17. The in vitro edible fat tissue of paragraph 16, wherein the population of insect precursor cells is a population of Manduca sexta precursor cells.
18. The in vitro edible fat tissue of any one of the paragraphs 15-17, wherein the scaffold is a fungal scaffold.
19. The in vitro edible fat tissue of paragraph 18, wherein the fungal scaffold is a decellularized mycelium scaffold.
20. The in vitro edible fat tissue of any one of paragraphs 15-19, wherein the lipid composition is an oil emulsion.
21. The in vitro edible fat tissue of paragraph 19, wherein the lipid composition is a seed oil emulsion.
22. An in vitro method of making edible fat tissue, the method comprising:
   seeding a population of insect precursor cells into a container; and
   contacting the population of insect precursor cells with a cell media containing a lipid composition, thereby accumulating lipids in the population of insect precursor cells and producing therefrom an edible fat tissue.

### EXAMPLES

### Example 1. Cell Isolation and Culture

M. sexta embryonic cells (i.e., Ms PCs) were isolated as described previously⁸. Briefly, M. sexta eggs were staged at 19-22 hours, sterilized with 50% sodium hypochlorite solution for 10 minutes, rinsed with M3+BPYE growth media (Shields and Sang M3 media (S8398, Sigma-Aldrich, St. Louis, MO) supplemented with 0.5 g/L potassium bicarbonate, 2.5 g/L bactopeptone, 1 g/L yeast extract and 10 (vol./vol.) % heat inactivated fetal bovine serum), 500 ng/ml methoprene (33375, Sigma-Aldrich), 1% antibiotic-antimycotic (15062, ThermoFisher) and 100 mg/mL primocin (NC9141851, Fisher Scientific) and homogenized⁵⁰. The homogenate was filtered, centrifuged (380×g for 10 minutes) twice to rinse and the cell pellet was resuspended in plating media (growth media supplemented with 1.14 mg/mL ethylene glycol-bis(2-aminoethyl)-N,N,N9,N9-tetraacetic acid). Cells were plated at 220,000/cm² and incubated for two hours at 27° C. in a non-humidified incubator without carbon dioxide gas exchange. Culture vessels were placed at a shaker plate for 10 minutes, non-adherent cells were aspirated, and cells were refreshed with growth media before returning the vessels to the incubator. Cells were allowed to grow undisturbed for two weeks before passaging and characterizing. Cells were routinely subcultured every 7 days in media without antibiotics at 90% confluency and seeded at 100,000 cells/cm². D. melanogaster embryonic cells (i.e., Dm PCs) (EF4005, Kerafast, Boston, MA) were also cultured in M3+BPYE growth media. Dm PCs were routinely seeded at 50,000 cells/cm², passaged at 90% confluency, replenished with fresh media once per week and incubated at 25° C. in a non-humidified incubator without carbon dioxide gas exchange.

### Example 2. Proliferation Assay of Cells

Cell growth was quantified by a CyQuant Cell Proliferation Assay (C7026, ThermoFisher). Ms PCs were seeded in triplicate at 25,000, 50,000 or 100,000 cells/cm² in 96-well plates. On days 1, 3 and 5, media was aspirated, and plates were frozen at −80 deg. C. At the time of assay, plates were thawed, and cells were incubated for 5 minutes in the dark with CyQuant working solution. Fluorescence was quantified at 480 nm excitation and 520 nm emission on a microplate reader.

### Example 3. Cryopreservation of Cells

Ms PCs were expanded in T25 and T75 cell culture flasks. When cells reached 90% confluency, cells were harvested by trypsinization for 5 minutes at 27° C. Cells were centrifuged at 380×g for 5 minutes and resuspended at 9E6 cells/mL in freezing media (i.e., 60% growth media, 30% heat inactivated fetal bovine serum and 10% dimethyl sulfoxide). Cells were transferred to cryovials and frozen in a cryobowl at −80° C. overnight before relocation to long-term liquid nitrogen storage. Cells were thawed by warming in a room temperature water bath for two minutes, diluting in growth media and centrifuging before counting, resuspending and seeding at 100,000 viable cells/cm². Cells were monitored for 10 days post-cryopreservation and passaged after 5 days. Cell viability and aggregation was determined with an automated cell counter (NC-200™, Chemometec, Denmark). Cell aggregation is defined as the percentage of cells within aggregates of five or more cells.

### Example 4. Mycoplasma Test

Mycoplasma presence was investigated via a MycoAlert assay (LT07, Lonza, Basel, Switzerland). Ms PC cell suspension was centrifuged at 380×g for 5 minutes and supernatant was transferred into a 96-well plate. Samples, negative controls and positive controls were incubated with a reagent solution for 5 minutes and luminescence was measured on a microplate reader. Samples were then incubated with a substrate solution for 10 minutes and luminescence was measured again. The second reading was divided by the first reading to generate the luminescence ratio. Samples, negative controls and positive controls were measured in duplicate.

### Example 5. Measurement of Lipid Accumulation

To screen for lipid accumulation in Ms PCs, cells were seeded at 100,000 cells/cm² in 48-well plates. After 24 hours in culture, media was refreshed with growth media supplemented with variable concentrations (0, 0.01, 0.1, 1 mM) of water-soluble oleic acid (01257, Sigma-Aldrich) or Intralipid (I141, Sigma-Aldrich). After seven days in culture, cells were stained with BODIPY (D3922, Fisher Scientific) at a 1:2,000 dilution and NucBlue (R37605, Fisher Scientific) or fixed and stained with Oil Red O (O0625, Sigma-Aldrich). Cells were imaged on a fluorescence microscope (BZ-X800, Keyence). To quantify lipid accumulation, Oil Red O dye from each condition was eluted with isopropanol and absorbance (500 nm) was measured on a microplate reader. To investigate lipid accumulation over time, cells were treated with 0.1 mM of Intralipid and stained with BODIPY and NucBlue on days 1, 4, 7 and 14.

### Example 6. Lipid Extraction from Cells

Ms PCs (p7) were seeded at 100,000/cm² into control or treated conditions. After 48 hours, 0.1 mM intralipid was added to the treated cells. Media for all cells was refreshed after 7 days. After 14 days, cells were harvested, and lipids were extracted via methyl tert-butyl ether (MTBE) extraction as previously described⁵¹. Briefly, cells were detached, counted, and divided into 2E6 cells per sample. Samples were resuspended and vortexed with methanol before adding MTBU and incubating for 1 hour. Water was added, samples vortexed, and centrifuged at 10,000×g for 5 minutes. The upper organic phase was removed, and MTBE extraction repeated. Double-extracted MTBE phases were dried under liquid nitrogen and stored at −20° C.

### Example 7. Lipidomic Analysis

Lipids were extracted as described above. Untargeted lipidomic profiling (n=5) was performed via high resolution liquid chromatography with tandem mass spectrometry (Thermo Scientific QExactive Plus/HF Orbitrap HR-LC-MS/MS) at the Mass Spectrometry Core at Beth Israel Deaconess Medical Center in Boston, Massachusetts. Data processing was performed in Python (v 3.8.5). Python's pandas (v 1.2.3) was used to organize the data for manipulation. The lipids were binned according to their classification (e.g., phospholipids, triglycerides). Within each classification, the lipid peak areas of the associated fatty acids were summed to determine total fatty acid area. The fatty acids were sorted as saturated, monounsaturated, or polyunsaturated.

### Example 8. Neutral Lipid Assay

Lipids were extracted as described herein. Neutral lipids were quantified with a Neutral Lipid Assay Kit (ab242307, Abcam) per manufacturer's instructions. Briefly, samples were diluted in a 2:1 methanol to chloroform mixture. Samples were incubated at 55° C. for 30 minutes until dry, briefly cooled at 4° C., rehydrated in isopropanol and incubated for 15 minutes with Fluorometric reagent. Fluorescence was quantified at 490 nm excitation and 585 nm emission on a microplate reader. A standard curve was prepared using lipid standards to estimate lipid values per sample.

### Example 9. Mycelia Decellularization & Scaffold Preparation

Excell™ Mycelium Scaffolds in uniform sheets of 3-6 mm thickness was generously donated by Ecovative Design (Green Island, NY). Mycelia were sectioned with a 6-8 mm biopsy punch to generate cylindrical samples of 6-8 mm diameter and 3-6-8 mm height. For decellularization, samples were submerged in 70% ethanol for 1 hour on an orbital shaker at 100 rpm. Samples were subsequently submerged in deionized water (24 hours), 1% sodium dodecyl sulfate (24 hours), 0.1% Tween 20 in 10% sodium hypochlorite bleach, deionized water (24 hours) and lastly 10 mM Tris buffer, pH 9.0 (24 hours). Samples were removed from the solution, frozen at −20° C. for at least 12 hours and lyophilized for 48 hours. Samples were sterilized by ethylene oxide prior to cell culture.

### Example 10. Sterilization Screen

To compare sterilization methods, mycelium samples were either left untreated, soaked in 70% ethanol overnight, soaked in 20% bleach overnight, autoclaved or exposed to ethylene oxide gas. Sterility was screened by incubating the scaffolds in basal media at 37° C. for two weeks and observing signs of visible contamination under a microscope.

### Example 11. DNA Quantification

DNA content in control and decellularized mycelium was quantified by a CyQuant assay (C7026, ThermoFisher Scientific, Waltham, MA). Mycelium samples frozen in liquid nitrogen and grinded into a powder with a mortar and pestle. Samples were suspended in cell lysis buffer and CyQuant reagent, centrifuged at 380×g for 5 minutes and supernatant was analyzed on a microplate reader at 480 nm excitation and 520 nm emission.

### Example 12. 3D Lipid Accumulation

Mycelium scaffolds were decellularized and sterilized as described above. Ms PCs were seeded at 6E6 cells per scaffold and allowed to adhere for 24 hours. For mechanical testing, Ms PCs were seeded at approximately 15M cells per scaffold (exact seeding density is unknown because not all cells adhered to the scaffold). Non-adherent cells were removed, and media refreshed with either growth media (control) or growth media supplemented with 0.1 mM Intralipid (treated). A 50% media change was performed after 7 days, and after 14 days scaffolds were either fixed for BODIPY/DAPI staining or used for mechanical testing. For BODIPY/DAPI staining, cell-loaded scaffolds were stained with BODIPY (D3922, Fisher Scientific) at a 1:2,000 dilution for 30 minutes. Next, they were fixed overnight in 4% paraformaldehyde, submerged in 15% sucrose (24 hours), 30% sucrose (24 hours) and Optimal Cutting Temperature (OCT) compound (24 hours). After fixation scaffolds were embedded in OCT in cryomolds, and sectioned with a Cryostat (CM1950, Leica, Wetzlar, Germany) at 15 mm. Cryosectioned scaffolds were mounted on a glass slide with Mounting Medium with DAPI (ab 104139, Abcam, Cambridge, United Kingdom) and imaged on a fluorescence microscope (BZ-X800, Keyence, Osaka, Japan).

### Example 13. Fat Body Histology

Fat body tissue was dissected from M. sexta 2nd day 5th instar larva which were first anesthetized with ice and carbon dioxide. The fat body tissues were placed in 10% neutral buffered formalin for 24 hours and subsequently rinsed 3× with phosphate buffered saline. To prepare for cryosectioning, tissues were submerged in 15% sucrose (24 hours), 30% sucrose (24 hours) and Optimal Cutting Temperature (OCT) compound (24 hours). OCT-embedded tissues were transferred to cryomolds and frozen with dry ice. Tissues were sectioned at 15 mm with a Cryostat (CM1950, Leica) and transferred to microscope slides. Tissues were stained with either BODIPY™ (D3835, ThermoFisher) diluted 1:2,500 in phosphate buffered saline or phalloidin (A12381, ThermoFisher) diluted 1:100 in phosphate buffered saline and mounted with DAPI-containing mounting medium (ab 104139, Abcam). Slides were imaged on a fluorescence microscope (BZ-X800, Keyence). Images were edited via Fiji software.

### Example 14. Texture Analysis

Texture analysis (e.g., double compression testing) was performed with a Dynamic Mechanical Analyzer. Cylindrical scaffolds and cell-scaffold constructs were compressed to 10% of their original height (ranging between approximately 1.5-3 mm) over a time span of 30 seconds, for a total of two cycles. The elastic moduli were calculated by measuring the slope between 5 and 15% strain of the second compression.

### Example 15. Confocal Microscopy

Cell-laden scaffolds were stained with BODIPY 488 at 1:1,000 (D3922, Fisher Scientific) for 30 minutes, fixed with 4% paraformaldehyde for 2 hours at room temperature, rinsed with PBS, and stained for 15 minutes with DAPI in PBS at 1:1,000 (D1306, ThermoFisher). Scaffolds were imaged on a glass plate on a confocal microscope (TCS SP8, Leica).

### Example 16. Statistical Analysis

All experiments were performed in triplicate unless otherwise noted. Statistical analysis was performed with GraphPad Prism Version 9 software. Statistical significance was determined for p-values less than 0.05. Unless otherwise specified, ANOVA with post hoc Tukey were performed.

### REFERENCES

1. Datar, I. & Betti, M. Possibilities for an in vitro Meat Production System. Innovative Food Science and Emerging Technologies 11, 13-22 (2010).
2. Post, M. J. et al. Scientific, sustainability and regulatory challenges of cultured meat. Nature Food 1, 403-415 (2020).
3. Rubio, N. R., Fish, K. D., Trimmer, B. A. & Kaplan, D. L. Possibilities for Engineered Insect Tissue as a Food Source. Frontiers in Sustainable Food Systems 3, 24 (2019).
4. Drugmand, J.-C. Characterization of insect cell lines is required for appropriate industrial processes: Case Study of High-Five Cells for Recombinant Protein Production. Chemistry & . . . (2007).
5. Chan, L. C. L. & Reid, S. Development of Serum-Free Media for Lepidopteran Insect Cell Lines. in Baculovirus and Insect Cell Expression Protocols (ed. Murhammer, D.) 161-196 (Springer Science & Business Media, 2016). doi: 10.1007/978-1-4939-3043-2 8.
6. Rubio, N. R., Mccartney, N. E., Trimmer, B. A. & Kaplan, D. L. Biofabrication with insect cells. Trends in Entomology 16, 1-17 (2020).
7. Rubio, N., Fish, K., Trimmer, B. & Kaplan, D. In Vitro Insect Muscle for Tissue Engineering Applications. ACS Biomaterials Science & Engineering 5, 1071-1082 (2019).
8. Baryshyan, A. L., Woods, W., Trimmer, B. A. & Kaplan, D. L. Isolation and Maintenance-Free Culture of Contractile Myotubes from Manduca sexta Embryos. PLOS ONE 7, e31598 (2012).
9. Baryshyan, A. L., Domigan, L. J., Hunt, B., Trimmer, B. A. & Kaplan, D. A. Self-Assembled Insect Muscle Bioactuators with Long Term Function Under a Range of Environmental Conditions. Royal Society of Chemistry Advances 75, 39962-39968 (2014).
10. Fish, K. D., Rubio, N. R., Stout, A. J., Yuen, J. S. K. & Kaplan, D. L. Prospects and Challenges for Cell-Cultured Fat as a Novel Food Ingredient. Trends in Food Science and Technology 98, 53-67 (2020).
11. Hoshizaki, D. Fat body. in The Insects: Structure and Function (eds. Simpson, S. J. & Douglas, A. E.) (Cambridge University Press, 2013).
12. Akiduki, G. & Imanishi, S. Establishment of a lipid accumulation model in an insect cell line. Archives of Insect Biochemistry and Physiology 66, 109-121 (2007).
13. Easton, C. M. & Horwath, K. L. Characterization of primary cell cultures derived from fat body of the beetle, Tenebrio molitor, and the immunolocalization of a thermal hysteresis protein in vitro. Journal of Insect Physiology 40, 537-547 (1994).
14. Mehta, F., Theunissen, R. & Post, M. J. Adipogenesis from Bovine Precursors. in 111-125 (Humana Press, New York, NY, 2019). doi: 10.1007/978-1-4939-8897-6 8.
15. Ding, S.-T., Wang, J.-C. & Mersmann, H. J. Effect of unsaturated fatty acids on porcine adipocyte differentiation. Nutrition Research 23, 1059-1069 (2003).
16. Beller, M. et al. COPI Complex Is a Regulator of Lipid Homeostasis. PLOS Biology 6, e292 (2008).
17. Guo, Y. et al. Functional genomic screen reveals genes involved in lipid-droplet formation and utilization. Nature 453, 657-661 (2008).
18. Campuzano, S. & Pelling, A. E. Scaffolds for 3D Cell Culture and Cellular Agriculture Applications Derived From Non-animal Sources. Frontiers in Sustainable Food Systems 3, 38 (2019).
19. Campuzano, S., Mogilever, N. & Pelling, A. Decellularized Plant-Based Scaffolds for Guided Alignment of Myoblast Cells. bioRxiv 2020.02.23.958686 (2020) doi: 10.1101/2020.02.23.958686.
20. Gershlak, J. R. et al. Crossing kingdoms: Using decellularized plants as perfusable tissue engineering scaffolds. Biomaterials 125, 13-22 (2017).
21. Allan, S. J., Ellis, M. J. & Bank, P. A. De. Decellularized grass as a sustainable scaffold for skeletal muscle tissue engineering. Journal of Biomedical Materials Research Part A (2021) doi: 10.1002/JBM.A.37241.
22. Wiebe, M. G. Quorn Myco-protein-Overview of a successful fungal product. Mycologist 18, 1-4 (2004).
23. Islam, M. R., Tudryn, G., Bucinell, R., Schadler, L. & Picu, R. C. Morphology and mechanics of fungal mycelium. Scientific Reports 7, 13070 (2017).
24. Simsa, R. et al. Extracellular heme proteins influence bovine myosatellite cell proliferation and the color of cell-based meat. Foods 8, 1-19 (2019).
25. Igene, J. O. & Pearson, M. Role of phospholipids and triglycerides in warmed-over flavor development in meat model systems. Journal of Food Science 44, 1285-1290 (1979).
26. Huang, Y.-C., Li, H.-J., He, Z.-F., Wang, T. & Qin, G. Study on the flavor contribution of phospholipids and triglycerides to pork. Food Science and Biotechnology 2010 19:5 19, 1267-1276 (2010).
27. Arshad, M. S. et al. Ruminant meat flavor influenced by different factors with special reference to fatty acids. Lipids in Health and Disease 2018 17:1 17, 1-13 (2018).
28. Carta, G., Murru, E., Banni, S. & Manca, C. Palmitic Acid: Physiological Role, Metabolism and Nutritional Implications. Frontiers in Physiology 0, 902 (2017).
29. Whelan, J. & Fritsche, K. Linoleic Acid. Advances in Nutrition 4, 311 (2013).
30. Clemente, T. E. & Cahoon, E. B. Soybean Oil: Genetic Approaches for Modification of Functionality and Total Content. Plant Physiology 151, 1030 (2009).
31 Wood, J. D. Meat Composition and Nutritional Value. in Lawrie's Meat Science: Eighth Edition 635-659 (Elsevier, 2017). doi: 10.1016/B978-O-08-100694-8.00020-0.
32. Ogg, C. L. & Stanley-Samuelson, D. W. Phospholipid and triacylglycerol fatty acid compositions of the major life stages and selected tissues of the tobacco hornworn Manduca sexta. Comparative Biochemistry and Physiology-B Biochemistry and Molecular Biology 101, 345-351 (1992).
33. Chen, L. et al. Inactivation of Salmonella enterica and Enterococcus faecium NRRL B2354 on cumin seeds using gaseous ethylene oxide. Food Microbiology 94, 103656 (2021).
34 SADRUD-DIN, S. Y., HAKIM, R. S. & LOEB, M. Proliferation and differentiation of midgut epithelial cells from Manduca sexta, in vitro. http://dx.doi.org/10.1080/07924259.1994.9672418 26, 197-204 (2011).
35. Sadrud-Din, S., Loeb, M. J. & Hakim, R. S. In vitro differentiation of isolated stem cells from the midgut of Manduca sexta larvae. Journal of Experimental Biology 199, 319-325 (1996).
36. Eide, P. E., Caldwell, J. M. & Marks, E. P. ESTABLISHMENT OF TWO CELL LINES FROM EMBRYONIC TISSUE OF THE TOBACCO HORNWORM, MANDUCA SEXTA (L.). IN VITRO 11, (1975).
37. Munekata, P. E., Pateiro, M., López-Pedrouso, M., Gagaoua, M. & Lorenzo, J. M. Foodomics in meat quality. Current Opinion in Food Science 38, 79-85 (2021).
38. Chen, J. & Liu, H. Nutritional Indices for Assessing Fatty Acids: A Mini-Review. International Journal of Molecular Sciences 2020, Vol. 21, Page 5695 21, 5695 (2020).
39. FB, H. et al. Dietary fat intake and the risk of coronary heart disease in women. The New England journal of medicine 337, 1491-1499 (1997).
40. LH, K. et al. Diet and 20-year mortality from coronary heart disease. The Ireland-Boston Diet-Heart Study. The New England journal of medicine 312, 811-818 (1985).
41. European Food Safety Authority. Scientific Opinion on Dietary Reference Values for fats, including saturated fatty acids, polyunsaturated fatty acids, monounsaturated fatty acids, trans fatty acids, and cholesterol. EFSA Journal 8, 1-107 (2016).
42. Miura, K. et al. Relationship of dietary monounsaturated fatty acids to blood pressure: the international study of macro/micronutrients and blood pressure. Journal of hypertension 31, 1144 (2013).
43. Chilton, F. H. et al. Precision Nutrition and Omega-3 Polyunsaturated Fatty Acids: A Case for Personalized Supplementation Approaches for the Prevention and Management of Human Diseases. Nutrients 2017, Vol. 9, Page 1165 9, 1165 (2017).
44. Carnevale De Almeida, J. et al. Fatty acid composition and cholesterol content of beef and chicken meat in Southern Brazil. Revista Brasileira de Ciências Farmacêuticas Brazilian Journal of Pharmaceutical Sciences 42, (2006).
45. Tu, T. H., Kim, H., Yang, S., Kim, J. K. & Kim, J. G. Linoleic acid rescues microglia inflammation triggered by saturated fatty acid. Biochemical and Biophysical Research Communications 513, 201-206 (2019).
46. Ascherio, A. et al. Dietary fat and risk of coronary heart disease in men: cohort follow up study in the United States. BMJ 313, 84-90 (1996).
47. Choque, B., Catheline, D., Rioux, V. & Legrand, P. Linoleic acid: Between doubts and certainties. Biochimie 96, 14-21 (2014).
48. Whelan, J. & Fritsche, K. Linoleic Acid. Advances in Nutrition 4, 311-312 (2013).
49. Comley, K. & Fleck, N. The compressive response of porcine adipose tissue from low to high strain rate. International Journal of Impact Engineering 46, 1-10 (2012).
50. Baum, B. & Cherbas, L. Drosophila cell lines as model systems and as an experimental tool. in Drosophila: Methods and Protocols (ed. Dahmann, C.) vol. 420 391-424 (Humana Press, 2008).
51. S B, B. et al. A relative quantitative positive/negative ion switching method for untargeted lipidomics via high resolution LC-MS/MS from any biological source. Metabolomics: Official journal of the Metabolomic Society 13, (2017).

We claim:

1. An in vitro method of making edible fat tissue, the method comprising:
seeding a population of insect cells onto a scaffold, wherein the scaffold is two- or three-dimensional; and
contacting the population of insect cells with a cell media containing a free fatty acid composition or a lipid composition, thereby accumulating lipids in the population of insect cells and producing therefrom an edible fat tissue.

2. (canceled)

3. The method of claim 1, wherein the population of insect cells is a population of Lepidoptera cells or Manduca sexta precursor cells.

4. The method of claim 1, wherein the scaffold is a fungal scaffold.

5. The method of claim 4, wherein the fungal scaffold is a decellularized Mycelium scaffold.

6. The method of claim 1, wherein the scaffold is two-dimensional, and the seeding is performed at a two-dimensional seeding density of at least one cell/cm², at least 15,000 cells/cm², at least 20,000 cells/cm², or at least 25,000 cells/cm² and at most 1,000,000 cells/cm², at most 500,000 cells/cm², at most 250,000 cells/cm², or at most 200,000 cells/cm², or at a range at or between the aforementioned values.

7. The method of claim 1, wherein the scaffold is three-dimensional and the seeding is performed at a three-dimensional seeding density that is a three-dimensional equivalent to the two-dimensional seeding density of at least one cell/cm², at least 15,000 cells/cm², at least 20,000 cells/cm², or at least 25,000 cells/cm² and at most 1,000,000 cells/cm², at most 500,000 cells/cm², at most 250,000 cells/cm², or at most 200,000 cells/cm², or at a range at or between the aforementioned values, or is at a three-dimensional seeding density of at least one cell/cm³, at least 15,000 cells/cm³, at least 20,000 cells/cm³, or at least 25,000 cells/cm³ and at most 1,000,000 cells/cm³, at most 500,000 cells/cm³, at most 250,000 cells/cm³, or at most 200,000 cells/cm³, or at a range at or between the aforementioned values.

8. The method of claim 1, further comprising the step of: cryopreserving and thawing the population of insect cells seeded on the scaffold.

9. The method of claim 1, wherein a concentration of lipids in the cell media from the lipid composition ranges from 0.1 mM to 10 mM, wherein the lipid composition is an emulsion.

10. The method of claim 9, wherein the lipid composition is a seed oil emulsion.

11. (canceled)

12. The method of claim 1, further comprising the step of isolating a population of insect cells.

13. A composition of matter made from the method of claim 1.

14. A food product containing the composition of matter of claim 13.

15. An in vitro edible fat tissue comprising a population of insect cells seeded on a scaffold, wherein the population of insect cells have been enriched in lipid content by exposure to a free fatty acid composition or a lipid composition.

16. (canceled)

17. The in vitro edible fat tissue of claim 1615, wherein the population of insect cells is a population of Lepidoptera cells or Manduca sexta precursor cells.

18. The in vitro edible fat tissue of claim 15, wherein the scaffold is a fungal scaffold.

19. The in vitro edible fat tissue of claim 18, wherein the fungal scaffold is a decellularized mycelium scaffold.

20. The in vitro edible fat tissue of claim 15, wherein the lipid composition is an oil emulsion.

21. The in vitro edible fat tissue of claim 19, wherein the lipid composition is a seed oil emulsion.

22. An in vitro method of making edible fat tissue, the method comprising:
seeding a population of insect cells into a container; and
contacting the population of insect cells with a cell media containing a free fatty acid composition or a lipid composition, thereby accumulating lipids in the population of insect cells and producing therefrom an edible fat tissue.

23. (canceled)

24. The method of claim 10, wherein the lipid composition is a soybean oil emulsion.
